(19) United States
(12) Patent Application Publication
(10) Pub. No.: US 2023/0104800 A1
(43) Pub. Date: Apr. 6, 2023

(54) COMBINATION THERAPIES AND BIOMARKERS FOR TREATING CANCER

(71) Applicants: Xiang LI, Beijing (CN); Chun JIANG, Hillsborough, CA (US); Yiyou Chen, San Jose, CA (US)

(72) Inventors: Xiang LI, Beijing (CN); Chun JIANG, Hillsborough, CA (US); Yiyou Chen, San Jose, CA (US)

(21) Appl. No.: 17/797,609

(22) PCT Filed: Feb. 5, 2021
(86) PCT No.: PCT/US2021/016861

§ 371 Date: Aug. 4, 2022

(30) Foreign Application Priority Data
Feb. 7, 2020 (CN) PCT/CN2020/074515

(51) Int. Cl. A61K 31/497; A61K 31/675; A61K 31/704; A61K 31/475; A61K 31/573; A61K 31/337; A61K 31/357; A61K 31/565; A61K 31/427; A61K 31/407; A61K 38/50; A61P 35/00; A61K 31/395

(52) U.S. Cl. CPC A61K 31/497; A61K 31/675; A61K 31/704; A61K 31/475; A61K 31/573; A61K 31/337; A61K 31/357; A61K 31/565; A61K 31/427; A61K 31/407; A61K 38/50; A61P 35/00; A61K 31/5545

(57) ABSTRACT

Provided are methods of using an anti-cancer agent's cell cycle-related, cell-killing activity to identify it as an effective combination with YM155 monobromide for treating MYC-associated cancers, and related kits, compositions, methods of screening, and methods for treating cancer in a subject in need thereof.

### CROSS-REFERENCE TO RELATED APPLICATIONS

This application claims priority to PCT/CN2020/074515, filed Feb. 7, 2020, which is incorporated by reference in its entirety.

### BACKGROUND

### Technical Field

Embodiments of the present disclosure relate to methods of using an anti-cancer agent's cell cycle-related, cell-killing activity to identify it as an effective combination with YM155 monobromide for treating MYC-associated cancers, and related kits, compositions, methods of screening, and methods for treating MYC-associated cancer in a subject in need thereof.

### Description of the Related Art

YM155 monobromide is a small-molecule survivin inhibitor that induces the down-regulation of survivin and exhibits potent antitumor activity (see, e.g., Minematsu et al., Drug Metabolism and Disposition, 37:619-628, 2008). YM-155 exerts anti-tumor effects in various in vivo cancer models, including prostate, pancreatic, and lung cancer (see, e.g., Nakahara et al., Cancer Research 67:8014-8021, 2007; and Na et al., PLoS One 7(6), 2012).

However, there is a need in the art to identify effective anti-cancer combination therapies for use with YM155 monobromide, and also to identify cancer patients that will benefit most from treatment with such combination therapies.

### BRIEF SUMMARY

Embodiments of the present disclosure include methods for treating a MYC-associated cancer in a subject in need thereof, comprising administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject in combination with a second anti-cancer agent, wherein cancer cell-killing activity of the second anti-cancer agent occurs predominantly in the M phase or G1 phase of the cell cycle,

thereby treating the MYC-associated cancer in the subject in need thereof.

Certain embodiments comprise

(a) determining MYC expression level, MYC gene copy number, or MYC gene chromosomal location site, in a sample of cancer tissue from the subject; and

(b) administering YM155 monobromide [1-(2-Methoxyethyl)-2-methyl-4,9-dioxo (pyrazin-2-ylmethyl)-4,9-dihydro-1H-naphtho[2,3-d] imidazolium bromide], or an analog or derivative thereof, to the subject in combination with the second anti-cancer agent if MYC expression level or MYC gene copy number in the cancer tissue is increased relative to that of a MYC expression level reference or MYC gene copy number reference, or if MYC gene chromosomal location site in the cancer tissue is translocated relative to that of a MYC gene chromosomal location site reference.

Certain embodiments comprise administering to the subject a chemotherapeutic agent excluding (or other than) YM155 monobromide if MYC expression level or MYC gene copy number in the cancer tissue is not substantially increased relative to that of the MYC expression level reference or MYC gene copy number reference, or if MYC gene chromosomal location site in the cancer tissue is not translocated relative to that of the MYC gene chromosomal location site reference.

In certain embodiments, at least 60, 65, 70, 75, 80, 85, 90, or 95% of the cancer cell-killing activity of the second anti-cancer agent occurs in the M phase of the cell cycle, optionally wherein the second anti-cancer agent is selected from one or more of vinca alkaloids and taxanes, or optionally selected from one or more of CHOP (cyclophosphamide, doxorubicin, vincristine, and prednisone), vinblastine, vincristine, vinorelbine, cabazitaxel, docetaxel, paclitaxel, eribulin, estramustine, and ixabepilone. In certain embodiments, at least 60, 65, 70, 75, 80, 85, 90, or 95% of the cancer cell-killing activity of the second anti-cancer agent occurs in the G1 phase of the cell cycle, optionally wherein the second anti-cancer agent is selected from one or more of mitomycin, asparaginase, and pegaspargase.

In certain embodiments, the MYC expression level or MYC gene copy number in the cancer tissue is increased by about or at least about 1.5, 2, 3, 4, 5, 6, 7, 8, 9, or 10-fold relative to that of the MYC expression level reference or MYC gene copy number reference.

Some embodiments comprise determining MYC expression level or MYC gene copy number in the cancer tissue by Western blot, in situ hybridization (ISH), fluorescence in situ hybridization (FISH), enzyme-linked immunosorbent assay (ELISA), array comparative genome hybridization (aCGH), single nucleotide polymorphism (SNP) array, copy number variation (CNV) sequencing, or multiplex ligation-dependent probe amplification (MLPA). Some embodiments comprise determining MYC gene chromosomal location site in the cancer tissue by in situ hybridization (ISH), fluorescence in situ hybridization (FISH), next generation sequencing (NGS), or comparative genome hybridization (CGH). Some embodiments comprise obtaining the MYC expression level or MYC gene copy number reference from a database, or determining the MYC expression level or MYC gene copy number reference from a non-cancerous tissue from a control, optionally by Western blot, ISH, FISH, ELISA, aCGH, SNP array, CNV sequence, or MLPA. Some embodiments comprise obtaining the MYC gene chromosomal location site reference from a database, or determining the MYC gene chromosomal location site reference from a non-cancerous tissue from a control, optionally by ISH, FISH, NGS, or CGH.

Some embodiments comprise obtaining the sample of cancer tissue from the subject. In some embodiments, the sample of cancer tissue is a surgical sample, a biopsy sample, a pleural effusion sample, or an ascetic fluid sample obtained from the subject, optionally selected from one or more of lung, blood, breast, gastrointestinal (stomach, colon, rectal), ovarian, pancreatic, liver, bladder, cervical, neuronal, uterine, salivary gland, kidney, prostate, thyroid, or muscle tissue. In some embodiments, the subject is a human subject.

In some embodiments, the cancer is selected from one or more of neuroblastoma, carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), radiation-induced angiosarcoma, medulloblastoma, astrocytoma, glioblastoma multiforme, retinoblastoma, myeloma, leukemia, lymphoma (including Hodgkin's lymphoma and Non-Hodgkin's lymphoma such as diffuse large B-cell lymphomas), adenosquamous carcinoma, carcinosarcoma, mixed mesodermal tumor, teratocarcinoma), lung cancer (including non-small cell lung cancer, small cell lung cancer, adenocarcinoma, and squamous carcinoma of the lung), breast cancer (including metastatic breast cancer), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

In some embodiments, the MYC gene is selected from MYCN and MYCC. In some embodiments, the MYC gene is MYCN and the cancer is selected from neuroblastoma, small cell lung cancer, prostate cancer, alveolar rhabdomyosarcoma, medulloblastoma, glioblastoma multiforme, retinoblastoma, and breast cancer. In some embodiments, the MYC gene is MYCC and the cancer is selected from lung cancers, optionally non-small lung cell cancer, blood cancers, optionally leukemias and lymphomas such as diffuse large B-cell lymphomas, and sarcomas, optionally radiation-induced angiosarcomas. In some embodiments, YM155 and the second anti-cancer agent are administered separately or sequentially. In some embodiments, YM155 and the second anti-cancer agent are administered together at the same time.

Also included are methods of screening an anti-cancer agent for use in combination with YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] for treating a MYC-associated cancer in a subject, comprising

(a) contacting a population of cancer cells (optionally in vitro) with the anti-cancer agent, wherein MYC expression level or MYC gene copy number in the cancer cells is increased relative to that of a MYC expression level reference or a MYC gene copy number reference, or wherein a MYC gene chromosomal location site in the cancer cells is translocated relative to that of a MYC gene chromosomal location site reference;

(b) measuring the amount of live:dead cancer cells in the M phase, G1 phase, and/or S/G2 phase of the cell cycle;

(c) characterizing or identifying or selecting the anti-cancer agent as effective for use in combination with YM155 monobromide if the cancer cell-killing activity of the anti-cancer agent occurs predominantly in the M phase or G1 phase of the cell cycle, and characterizing anti-cancer agent as not effective for use in combination with YM155 monobromide if the cancer cell-killing activity of the anti-cancer agent occurs predominantly in the S/G2 phase of the cell cycle.

In some embodiments, the second anti-cancer agent is selected from one or more of chemotherapeutic agents, cancer immunotherapy agents, hormonal therapeutic agents, and kinase inhibitors, including combinations of the foregoing. In some embodiments, the chemotherapeutic agent is selected from one or more of an alkylating agent, an anti-metabolite, a cytotoxic antibiotic, a topoisomerase inhibitor (type 1 or type II), and an anti-microtubule agent. In some embodiments, the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);

the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);

the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;

the topoisomerase inhibitor is selected from one or more of camptothecin, irinotecan, topotecan, etoposide, doxorubicin, mitoxantrone, teniposide, novobiocin, merbarone, and aclarubicin; and/or

the anti-microtubule agent is selected from one or more of taxanes (optionally paclitaxel and docetaxel) and vinca alkaloids (optionally vinblastine, vincristine, vindesine, vinorelbine).

In some embodiments, the cancer immunotherapy agent is an antagonist of an inhibitory immune checkpoint molecule selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT); optionally wherein the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736); optionally wherein the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab; optionally wherein the antagonist is a CTLA-4 antagonist optionally selected from one or more of an antibody or antigen-binding fragment or small molecule that specifically binds thereto, ipilimumab, and tremelimumab.

In some embodiments, the cancer immunotherapy agent is an agonist of a stimulatory immune checkpoint molecule selected from one or more of OX40, CD40, Glucocorticoid-Induced TNFR Family Related Gene (GITR), CD137 (4-1BB), CD27, CD28, CD226, and Herpes Virus Entry Mediator (HVEM). In some embodiments, the cancer immunotherapy agent is a cytokine selected from one or more of interferon (IFN)-α, IL-2, IL-12, IL-7, IL-21, and Granulocyte-macrophage colony-stimulating factor (GM-CSF).

In some embodiments, the hormonal therapeutic agent is a hormonal agonist or a hormonal antagonist, optionally wherein the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog, optionally wherein the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib, optionally wherein the kinase inhibitor is a PI3 kinase inhibitor selected from one or more of alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147.

Certain embodiments comprise characterizing or identifying or selecting the anti-cancer agent as effective for use in combination with YM155 monobromide if at least 60, 65, 70, 75, 80, 85, 90, or 95% of the cancer cell-killing activity of the anti-cancer agent occurs in the M phase of the cell cycle.

Certain embodiments comprise characterizing or identifying or selecting the anti-cancer agent as effective for use in combination with YM155 monobromide if at least 60, 65, 70, 75, 80, 85, 90, or 95% of the cancer cell-killing activity of the anti-cancer agent occurs in the G1 phase of the cell cycle.

Certain embodiments comprise

(d) contacting a population of cancer cells (optionally in vitro) with YM155 in combination with the identified or selected anti-cancer agent from (c), wherein MYC expression level or MYC gene copy number in the cancer cells is increased relative to that of a MYC expression level reference or MYC gene copy number reference, or wherein a MYC gene chromosomal location site in the cancer cells is translocated relative to that of a MYC gene chromosomal location site reference;

(e) measuring tumor cell proliferation and/or tumor cell apoptosis in the population of cancer cells; and

(f) characterizing or identifying or selecting the anti-cancer agent as optimal for use in combination with YM155 monobromide if the combined cancer cell-killing activity of the anti-cancer agent and YM155 is significantly (optionally synergistically) increased relative to that of YM155 and the anti-cancer agent alone.

In some embodiments, the population of cancer cells is selected from one or more of neuroblastoma, carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), radiation-induced angiosarcoma, medulloblastoma, astrocytoma, glioblastoma multiforme, retinoblastoma, myeloma, leukemia, lymphoma (including Hodgkin's lymphoma and Non-Hodgkin's lymphoma such as diffuse large B-cell lymphomas), adenosquamous carcinoma, carcinosarcoma, mixed mesodermal tumor, teratocarcinoma), lung cancer (including non-small cell lung cancer, small cell lung cancer, adenocarcinoma, and squamous carcinoma of the lung), breast cancer (including metastatic breast cancer), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

In some embodiments, the MYC gene is selected from MYCN and MYCC. In some embodiments, the MYC gene is MYCN and the population of cancer cells is selected from neuroblastoma, small cell lung cancer, prostate cancer, alveolar rhabdomyosarcoma, medulloblastoma, glioblastoma multiforme, retinoblastoma, and breast cancer. In some embodiments, the MYC gene is MYCC and the population of cancer cells is selected from lung cancers, optionally non-small lung cell cancer, blood cancers, optionally leukemias and lymphomas such as diffuse large B-cell lymphomas, and sarcomas, optionally radiation-induced angiosarcomas.

Also included are patient care kits, comprising:

(a) means for measuring MYC expression level, MYC gene copy number, or MYC gene chromosomal location site, in a sample of tissue from a subject, including cancer tissue and non-cancerous tissue;

(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof; and

(c) a second anti-cancer agent, wherein cancer cell-killing activity of the second anti-cancer agent occurs predominantly in the M phase and/or G1 phase of the cell cycle.

In some embodiments, the means for measuring MYC expression level or MYC gene copy number comprise reagents for performing a diagnostic assay selected from one or more of Western blot, in situ hybridization (ISH), fluorescence in situ hybridization (FISH), enzyme linked immunosorbent assay (ELISA), array comparative genome hybridization (aCGH), single nucleotide polymorphism (SNP) array, copy number variation (CNV) sequencing, and multiplex ligation-dependent probe amplification (MLPA) on a human MYC gene.

In some embodiments, the means for measuring MYC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYC gene.

Certain kits comprise a MYC expression level reference or a MYC gene copy number reference value obtained from a database, or determined from a non-cancerous tissue from a control. Certain kits comprise a MYC gene chromosomal location site reference obtained from a database, or determined from a non-cancerous tissue from a control.

In some embodiments, the MYC gene is selected from MYCC and MYCN. In some embodiments, at least 60, 65, 70, 75, 80, 85, 90, or 95% of the cancer cell-killing activity of the second anti-cancer agent occurs in the M phase of the cell cycle, optionally wherein the second anti-cancer agent is selected from one or more of vinca alkaloids and taxanes, or optionally selected from one or more of CHOP (cyclophosphamide, doxorubicin, vincristine, and prednisone), vinblastine, vincristine, vinorelbine, cabazitaxel, docetaxel, paclitaxel, eribulin, estramustine, and ixabepilone. In some embodiments, at least 60, 65, 70, 75, 80, 85, 90, or 95% of the cancer cell-killing activity of the second anti-cancer agent occurs in the G1 phase of the cell cycle, optionally wherein the second anti-cancer agent is selected from one or more of mitomycin, asparaginase, and pegaspargase.

### BRIEF DESCRIPTION OF THE DRAWINGS

FIG. 1 shows the chemical structure of YM155 monobromide (CAS 781661-94-7).

FIG. 2 and FIG. 3 provide genomic information for the human MYC gene (see uswest.ensembl.org/Homo_sapiens/Location/View?db=core;g=ENSG00000136997;r=8:127735434-127742951).

FIG. 4 shows the location of the MYCN gene on the short (p) arm of chromosome 2 at position 24.3.

FIG. 5 illustrates the cell cycle-related fate of neuroblastoma cells following YM155 (PC-002) treatment.

FIGS. 6A-6C shows the cell cycle-related neuroblastoma-killing activity of YM155 (PC002) alone, CHOP alone, and the combination of YM155/CHOP. Shown are the relative percentage of dead and live neuroblastoma cells in the G1 phase (6A), the S/G2 phase (6B), and M phase (6C) following treatment, as indicated.

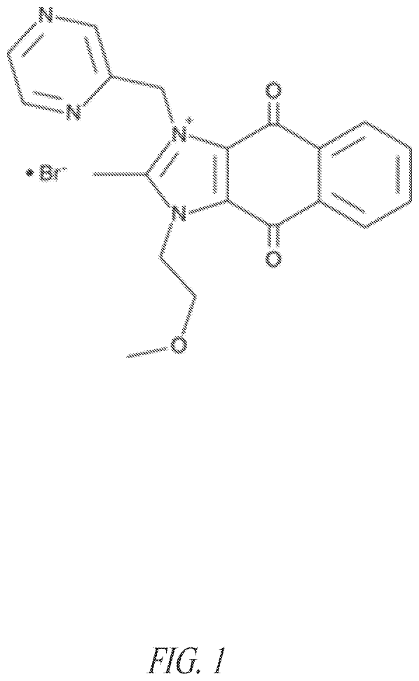

### DETAILED DESCRIPTION

The present disclosure relates to the unexpected discovery that the cancer cell-killing activity of YM155 occurs predominantly during the S/G2 phase(s) of the cell cycle. It further relates to the discovery that amplifications and/or translocations of the MYC gene in human malignancies associate with increased anti-cancer efficacy of the chemotherapeutic agent YM155 monobromide, and can thus be used as biomarkers to optimize cancer therapy by that agent and others (see, for example, PCT/US2019/046124, incorporated by reference in its entirety). Thus, as illustrated in the Examples section, these discoveries evidence that optimal synergy in YM155 combination therapies can be achieved by using at least one agent with cancer cell-killing activity that occurs predominantly during other phases of the cell cycle, such as the M (mitosis) phase and/or G1 phase. Such YM155-based combination therapies and methods of screening for effective combinations could provide significant utility in the treatment of cancers, particularly MYC-associated cancers.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

An “antagonist” or “inhibitor” refers to biological structure or chemical agent that interferes with or otherwise reduces the physiological action of another molecule, such as a protein (e.g., survivin). In some instances, the antagonist or inhibitor specifically binds to the other molecule and/or a functional ligand of the other molecule. In some instances, the antagonist or inhibitor down-regulates the expression of the other molecule (e.g., survivin). Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The “half maximal inhibitory concentration” (or “IC₅₀”) is a measure of the potency of an agent in inhibiting a specific biological or biochemical function. This quantitative measure indicates how much of a particular agent (inhibitor) is needed to inhibit a given biological process (or component of a process, i.e. an enzyme, cell, cell receptor or microorganism) by half. The values are typically expressed as molar concentration. The concentration is commonly used as a measure of antagonist drug potency in pharmacological research. In some instances, IC₅₀ represents the concentration of an agent that is required for 50% inhibition in vitro. The IC₅₀ of an agent can be determined by constructing a dose-response curve and examining the effect of different concentrations of the agent on the desired activity, for example, inhibition of tumor cell proliferation, tumor-cell killing

An “increased” or “enhanced” amount is typically a “statistically significant” amount, and may include an increase that is about or at least about 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 50, 60, 70, 80, 90, 100, 200, 300, 400, 500, or 1000 fold, or about or at least about 5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, relative to that of a reference or control (including all integers and ranges in between). A “decreased” or “reduced” amount is typically a “statistically significant” amount, and may include a decrease that is about or at least about 1.2, 1.4, 1.6, 1.8, 2, 3, 4, 5, 6, 7, 8, 9, 10, 15, 20, 30, 50, 60, 70, 80, 90, 100, 200, 300, 400, 500, 1000 fold, or about or at least about 5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, relative to that of a reference or control (including all integers and ranges in between).

The term “polynucleotide” and “nucleic acid” includes mRNA, RNA, cRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Embodiments of the present disclosure include methods for treating a MYC-associated cancer in a subject in need thereof, comprising administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject in combination with a second anti-cancer agent, wherein cancer cell-killing activity of the second anti-cancer agent occurs predominantly in the M phase or G1 phase of the cell cycle, thereby treating the MYC-associated cancer.

The feature “MYC-associated cancer” refers to a cancer (e.g., cell or tissue) in which MYC gene copy number in the cancer is increased relative to that of a MYC gene copy number reference, and/or a cancer in which MYC gene chromosomal location site in the cancer is translocated relative to that of a MYC gene chromosomal location site reference, as described herein.

A “second anti-cancer agent” refers essentially to any other anti-cancer agent except YM155, which predominantly kills cancer cells in the M phase and/or G1 phase of the cell cycle. General examples of anti-cancer agents include chemotherapeutic agents, cancer immunotherapy agents, hormonal therapeutic agents, and/or kinase inhibitors, including combinations of more than one anti-cancer agent. The feature “predominantly” refers to about 50% or more, for example, wherein at least 50, 60, 65, 70, 75, 80, 85, 90, or 95% of the cancer cell-killing activity of the second anti-cancer agent occurs in the M phase and/or G1 phase of the cell cycle.

In certain embodiments, at least 60, 65, 70, 75, 80, 85, 90, or 95% of the cancer cell-killing activity of the second anti-cancer agent occurs in the M phase of the cell cycle. General examples of such agents include mitotic agents such as vinca alkaloids and taxanes. Particular examples include vinblastine, vincristine, vinorelbine, cabazitaxel, docetaxel, paclitaxel, eribulin, estramustine, and ixabepilone. Also included is the combination therapy CHOP (cyclophosphamide, doxorubicin, vincristine, and prednisone; as described, for example, in van Agthoven et al., Hematol J. 4(6):399-409, 2003), and others. Thus, certain embodiments comprise administering YM155 in combination with one or more of the foregoing anti-cancer agents such as CHOP.

In some embodiments, at least 60, 65, 70, 75, 80, 85, 90, or 95% of the cancer cell-killing activity of the second anti-cancer agent occurs in the G1 phase of the cell cycle. Particular examples include mitomycin, asparaginase, and pegaspargase. Thus, certain embodiments comprise administering YM155 in combination with one or more of such agents.

Certain embodiments comprise the steps of (a) determining MYC expression level, MYC gene copy number, or MYC gene chromosomal location site, in a sample of cancer tissue from the subject; and (b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] to the subject if MYC expression level or MYC gene copy number in the cancer tissue is increased relative to that of a MYC expression level reference or MYC gene copy number reference, or if MYC gene chromosomal location site in the cancer tissue is translocated relative to that of a MYC gene chromosomal location site reference, thereby treating cancer in the subject in need thereof. Certain embodiments include administering to the subject a chemotherapeutic agent excluding (or other than) YM155 monobromide if MYC expression level or MYC gene copy number in the cancer tissue is not substantially increased (e.g., the same or less than about 1.1 fold increase) relative to that of the MYC expression level reference or MYC gene copy number reference, or if MYC gene chromosomal location site in the cancer tissue is not translocated relative to that of the MYC gene chromosomal location site reference.

The “MYC gene” or “MYC oncogene” refers to a family of proto-oncogenes that encode transcription factors, examples of which include c-Myc (also MYCC) and N-myc (also MYCN).

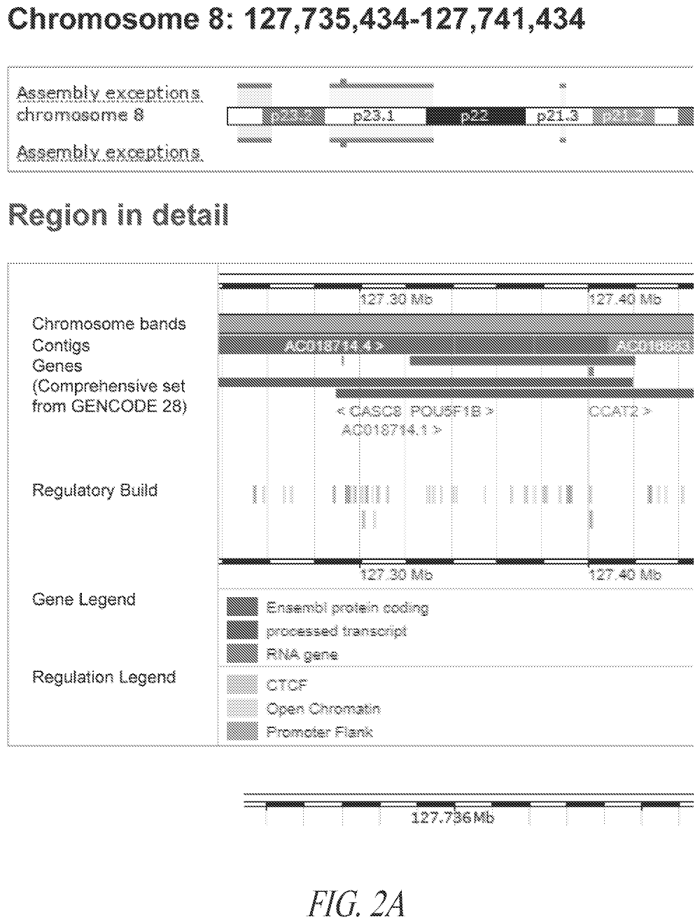

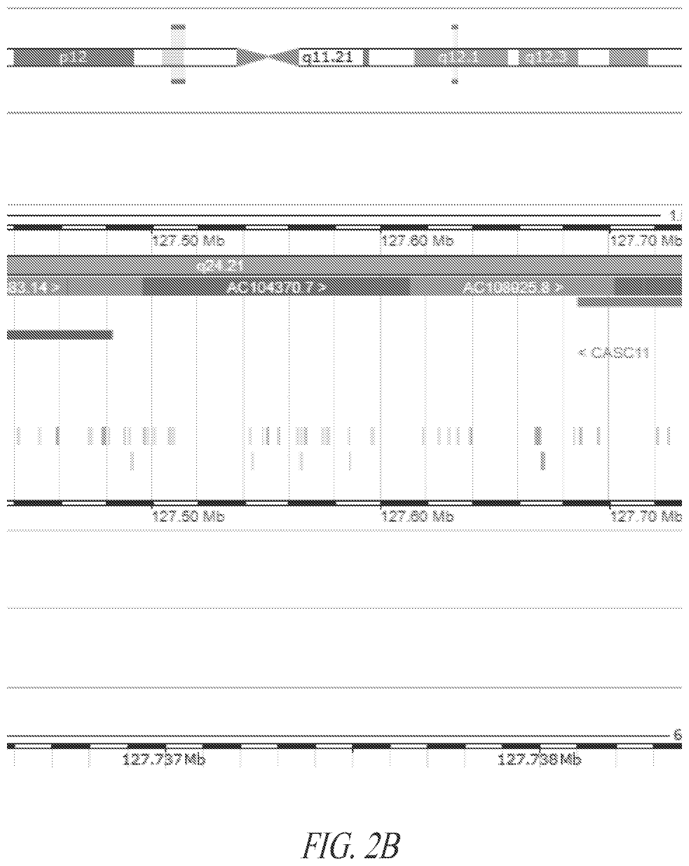

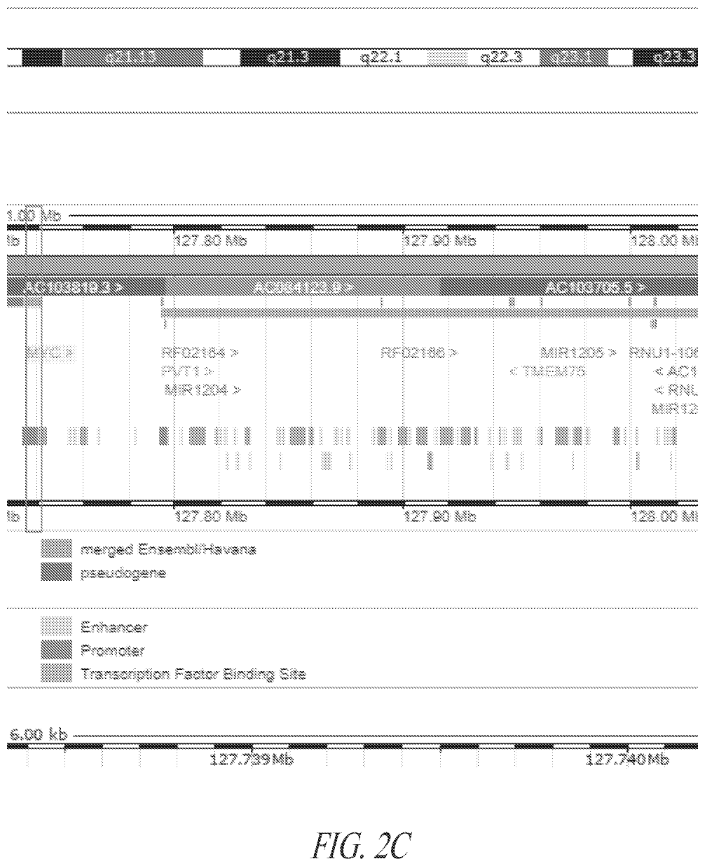

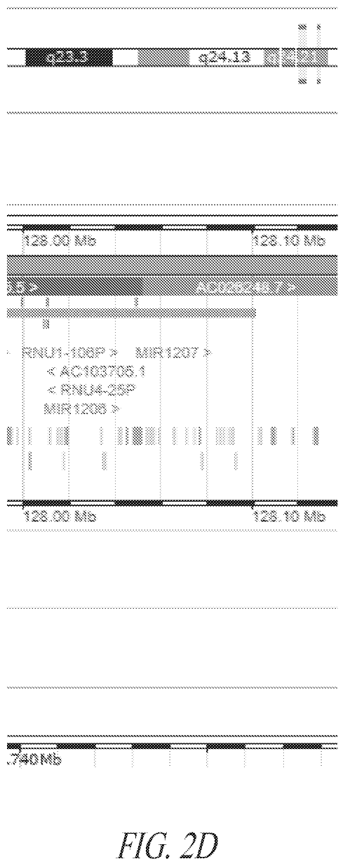

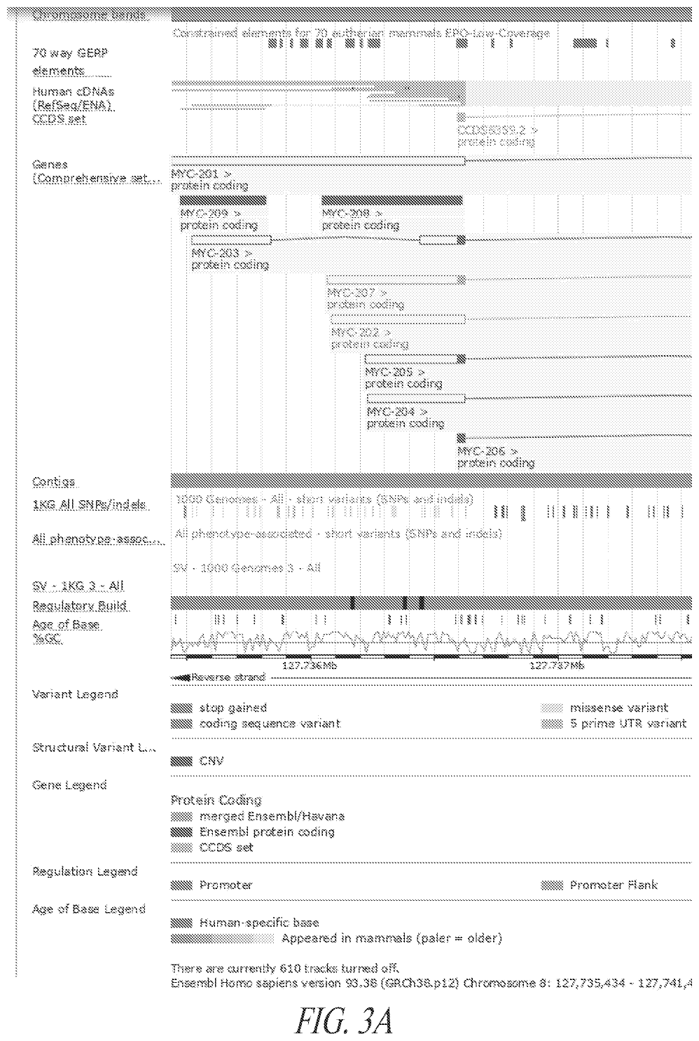

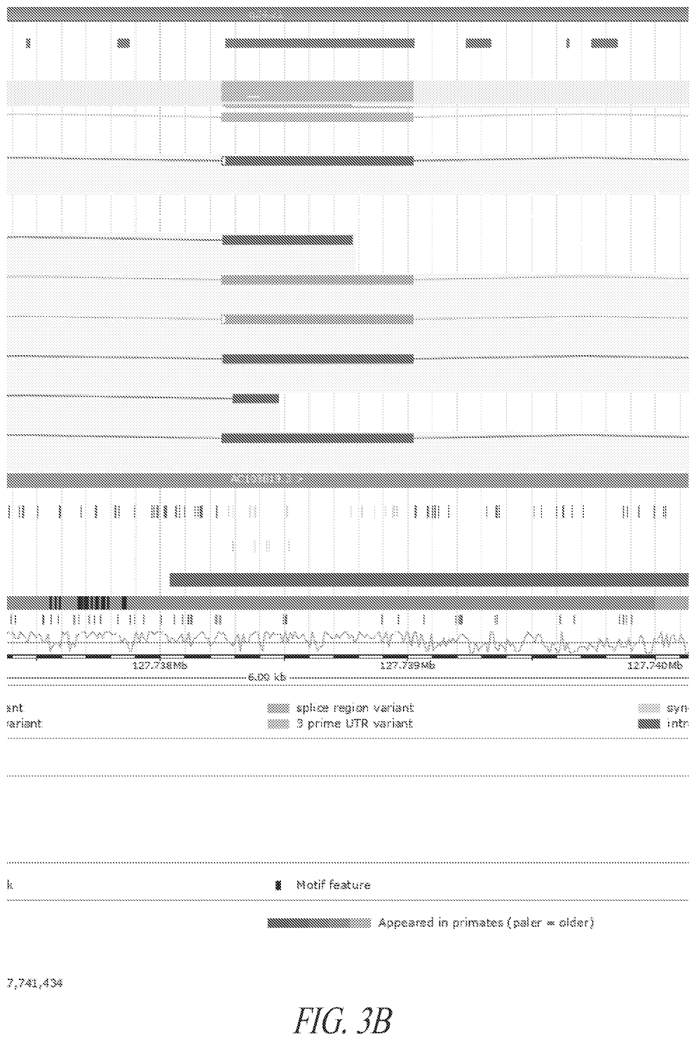

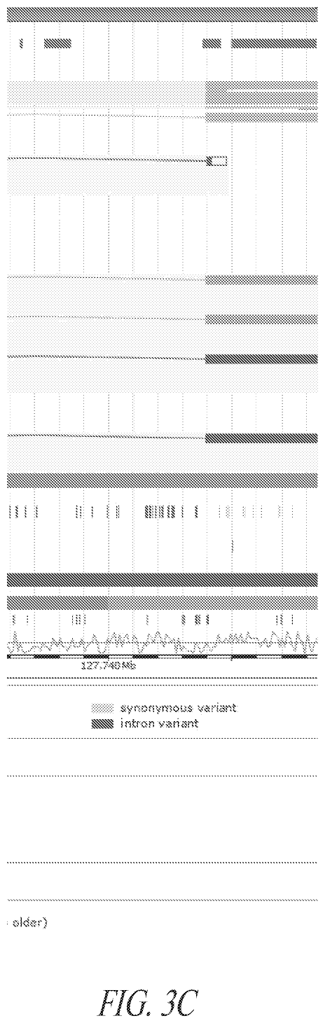

The MYCC gene encodes a nuclear phosphoprotein that plays a role in cell cycle progression, apoptosis, and cellular transformation. The encoded protein forms a heterodimer with the related transcription factor MAX. This complex binds to the E box DNA consensus sequence and regulates the transcription of specific target genes. There is evidence to show that translation initiates both from an upstream, in-frame non-AUG (CUG) and a downstream AUG start site, resulting in the production of two isoforms with distinct N-termini. In the human genome, the MYCC gene is located on chromosome 8:127, 735, 434-127, 741, 434, forward strand (see, e.g., FIG. 2 and FIG. 3; and Gene: MYC ENSG00000136997).

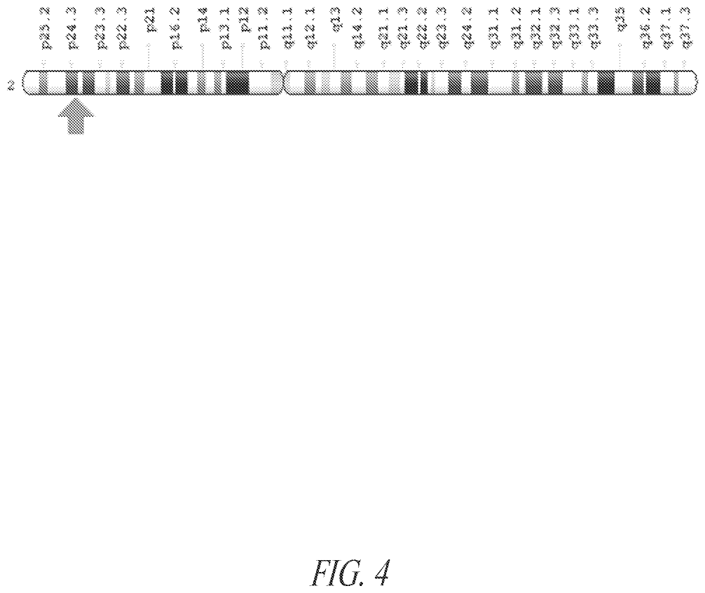

The MYCN gene encodes a protein with a basic helix-loop-helix (bHLH) domain. It is located in the cell nucleus and dimerizes with another bHLH protein to bind DNA. MYCN is over-expressed in a number of different types of cancer, including, for example, neuroblastoma, rhabdomyosarcoma, medulloblastoma, astrocytoma, glioblastoma, retinoblastoma, prostate cancer, breast cancer, Wilms' tumour, and small cell lung cancer (see, for example, Beltran, Mol Cancer Res. 12:815-822, 2014). Indeed, MYCN amplification is an adverse prognostic factor in neuroblastoma. The amplicon (material co-amplified with MYCN) varies among subjects, and in certain instances includes, for example, the DDX1 gene. In some instances, MYCN amplification correlates with a 1p36 deletion and a gain of chromosome 17q. In the human genome, the MYCN gene is located on the short (p) arm of chromosome 2 at position 24.3 (Cytogenetic Location at 2p24.3; Molecular Location at base pairs 15,940,438 to 15,947,007 on chromosome 2; see also FIG. 4).

Thus, in certain embodiments, the MYC gene is selected from MYCC and MYCN. “YM155 monobromide” refers to the small molecule [1-(2-Methoxyethyl)-2-methyl-4,9-dioxo-3-(pyrazin-2-ylmethyl)-4,9-dihydro-1H-naphtho[2,3-d] imidazolium bromide], having the molecular formula C₂₀H₁₉N₄O₃.Br, and the CAS Number 781661-94-7, and includes pharmaceutically-acceptable salts and acids thereof. Also included are biologically-active or equivalent analogs and/or derivatives of YM155 monobromide.

As noted above, in some instances, the MYC expression level or MYC gene copy number in the cancer tissue is increased relative to that of the MYC gene copy number reference. In particular embodiments, the MYC expression level or MYC gene copy number in the cancer tissue is increased by a statistically significant amount relative to that of the MYC expression level reference or MYC gene copy number reference. In some embodiments, the MYC expression level or MYC gene copy number in the cancer tissue is increased by about or at least about 1.2, 1.3, 1.4, 1.5, 1.6, 1.7, 1.8, 1.9, 2, 2.5, 3, 3.5, 4, 4.5, 5, 6, 7, 8, 9, or 10-fold (or more) relative to that of the MYC expression level reference or MYC gene copy number reference.

The MYC expression level or MYC gene copy number in the cancer tissue can be determined by any variety of methods. For example, in some embodiments, the MYC expression level is determined by Western blot, in situ hybridization (ISH), fluorescence in situ hybridization (FISH), enzyme-linked immunosorbent assay (ELISA), or other method for evaluating expression levels. In some embodiments, the MYC gene copy number is determined by array comparative genome hybridization (aCGH), single nucleotide polymorphism (SNP) array, copy number variation (CNV) sequencing, or multiplex ligation-dependent probe amplification (MLPA). Certain embodiments thus include the step of determining or detecting copy number of a MYC gene in a sample of cancer tissue from a subject in need thereof. Also included is the step of comparing the copy number of a MYC gene in a sample of cancer tissue relative to that of a MYC gene copy number reference.

The MYC gene chromosomal location site in the cancer tissue can be determined by any variety of methods. For example, in some embodiments, the MYC gene chromosomal location site in the cancer tissue is determined by in situ hybridization (ISH), fluorescence in situ hybridization (FISH), next generation sequencing (NGS), or comparative genome hybridization (CGH). Certain embodiments thus include the step of determining or detecting the MYC gene chromosomal location site in a sample of cancer tissue from a subject in need thereof. Also included is the step of comparing the MYC gene chromosomal location site in the cancer tissue relative to that of a MYC gene chromosomal site reference.

CGH refers to a molecular cytogenetic method for analyzing copy number variations (CNVs) relative to ploidy level in the DNA of a test sample compared to a reference sample, without the need for culturing cells. This technique allows quick and efficient comparisons between two genomic DNA samples arising from two sources, which are most often closely related, because it is suspected that they contain differences in terms of either gains or losses of either whole chromosomes or subchromosomal regions (a portion of a whole chromosome). The technique was originally developed for the evaluation of the differences between the chromosomal complements of solid tumor and normal tissue (see, e.g., Kallioniemi et al., Science. 258 (5083): 818-821, 1992). The use of DNA microarrays in conjunction with CGH techniques has led to the development of a more specific form of array CGH (aCGH), allowing for a locus-by-locus measure of CNV with increased resolution as low as 100 kilobases (see, e.g., Pinkel, Annu Rev Genom Hum Genet. 6:331-354, 2005). CNV is a prevalent form of critical genetic variation that leads to an abnormal number of copies of large genomic regions in a cell, and high-resolution sequence data can be analyzed by next-generation sequencing (NGS) to identify the same (see, e.g., Zhao et al., BMC Bioinformatics. 14 Suppl 11:S1, 2013). MLPA refers to a variation of the multiplex polymerase chain reaction that permits amplification of multiple targets with only a single primer pair (see, e.g., Schouten et al., Nucleic Acids Res. 30 (12): e57, 2002). In situ hybridization (ISH) and fluorescent in situ hybridization (FISH) refer to a type of hybridization that uses a labeled complementary DNA, RNA or modified nucleic acids strand (i.e., probe) to localize a specific DNA or RNA sequence in a portion or section of tissue (in situ) (see, e.g., Parra & Windle, Nature Genetics. 5:17-21, 1993; and Gall & Pardue, PNAS USA. 63: 378-383, 1969). Thus, in some instances, the methods and kits described herein employ any one or more of the foregoing techniques and/or comprise reagents for performing the same.

Examples of a “reference” (e.g., a MYC expression level “reference”, a MYC gene copy number “reference”, a MYC gene chromosomal site “reference”) include a value or amount or location obtained from a database, for example, a value or amount of a “wild-type” MYC expression level reference or gene copy number, or a “wild-type” MYC gene chromosomal location site (see, e.g., FIG. 2 and FIG. 3 for a human MYCC gene chromosomal site reference; and FIG. 4 for a human MYCN gene chromosomal site reference). A “reference” also includes a value or amount or location obtained from a non-cancerous tissue from one or more controls, for example, one or more healthy or non-cancerous control subjects (e.g., a population of healthy or non-cancerous control subjects), or one or more corresponding non-cancerous control tissues from the subject being tested. Typically, a “corresponding” non-cancerous control tissue is obtained from the same type of tissue as the cancer tissue being tested. As with the cancer tissue, the MYC gene copy number reference from a non-cancerous control can be determined by any variety of methods, including, for example, by aCGH, SNP array, CNV sequence, and/or MLPA (supra) Similarly, the MYC gene chromosomal location site reference from a non-cancerous control can be determined by any variety of methods, including, for example, ISH, FISH, NGS, and/or CGH (supra).

In some embodiments, the sample of cancer tissue (or non-cancerous control tissue) is a surgical sample, a biopsy sample, a pleural effusion sample, or an ascetic fluid sample from the subject. Particular examples of samples of cancer tissues (or non-cancerous control tissues) include lung, blood, breast, gastrointestinal (stomach, colon, rectal), ovarian, pancreatic, liver, bladder, cervical, neuronal, uterine, salivary gland, kidney, prostate, thyroid, or muscle tissues. Certain embodiments include the step of obtaining the sample of cancer tissue (or non-cancerous control tissue) from the subject, for example, prior to determining MYC gene copy levels or MYC gene chromosomal location site.

In some embodiments, the subject is a human subject.

As noted above, certain embodiments include administering to the subject an anti-cancer agent excluding (or other than) YM155 monobromide if the subject is characterized as non-responsive to YM155 monobromide therapy, for example, if the MYC expression level or MYC gene copy number in the cancer tissue is not substantially increased relative to that of the MYC gene copy number reference, or if the MYC gene chromosomal location site in the cancer tissue is not translocated relative to that of the MYC gene chromosomal location site reference. Exemplary anti-cancer agents (other than YM155 monobromide) for administering to a subject characterized as non-responsive to YM155 monobromide therapy include small molecules such as cytotoxic, chemotherapeutic, and anti-angiogenic agents, for instance, those that have been considered useful in the treatment of various cancers. General classes of anti-cancer agents include, without limitation, alkylating agents, anti-metabolites, anthracyclines, anti-tumor antibiotics, platinums, type 1 topoisomerase inhibitors, type II topoisomerase inhibitors, vinca alkaloids, and taxanes.

Specific examples of anti-cancer agents for administering to a subject characterized as non-responsive to YM155 monobromide therapy include chlorambucil, cyclophosphamide, cilengitide, lomustine (CCNU), melphalan, procarbazine, thiotepa, carmustine (BCNU), enzastaurin, busulfan, daunorubicin, doxorubicin, gefitinib, erlotinib idarubicin, temozolomide, epirubicin, mitoxantrone, bleomycin, cisplatin, carboplatin, oxaliplatin, camptothecins, irinotecan, topotecan, amsacrine, etoposide, etoposide phosphate, teniposide, temsirolimus, everolimus, vincristine, vinblastine, vinorelbine, vindesine, CT52923, and paclitaxel, and pharmaceutically acceptable salts, acids or derivatives of any of the above. Additional examples of anti-cancer agents include imatinib, crizotinib, dasatinib, sorafenib, pazopanib, sunitinib, vatalanib, geftinib, erlotinib, AEE-788, dichoroacetate, tamoxifen, fasudil, SB-681323, and semaxanib (SU5416) (see Chico et al., Nat Rev Drug Discov. 8:829-909, 2009).

Further examples of anti-cancer agents for administering to a subject characterized as non-responsive to YM155 monobromide therapy include alkylating agents such as thiotepa, cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vinde sine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and docetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above.

Additional examples of anti-cancer agents for administering to a subject characterized as non-responsive to YM155 monobromide therapy include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

Further examples of anti-cancer agents for administering to a subject characterized as non-responsive to YM155 monobromide therapy include anti-cancer antibodies such as 3F8, 8H9, abagovomab, adecatumumab, afutuzumab, alemtuzumab, alacizumab (pegol), amatuximab, apolizumab, bavituximab, bectumomab, belimumab, bevacizumab, bivatuzumab (mertansine), brentuximab vedotin, cantuzumab (mertansine), cantuzumab (ravtansine), capromab (pendetide), catumaxomab, cetuximab, citatuzumab (bogatox), cixutumumab, clivatuzumab (tetraxetan), conatumumab, dacetuzumab, dalotuzumab, detumomab, drozitumab, ecromeximab, edrecolomab, elotuzumab, enavatuzumab, ensituximab, epratuzumab, ertumaxomab, etaracizumab, farletuzumab, FBTA05, figitumumab, flanvotumab, galiximab, gemtuzumab, ganitumab, gemtuzumab (ozogamicin), girentuximab, glembatumumab (vedotin), ibritumomab tiuxetan, icrucumab, igovomab, indatuximab ravtansine, intetumumab, inotuzumab ozogamicin, ipilimumab (MDX-101), iratumumab, labetuzumab, lexatumumab, lintuzumab, lorvotuzumab (mertansine), lucatumumab, lumiliximab, mapatumumab, matuzumab, milatuzumab, mitumomab, mogamulizumab, moxetumomab (pasudotox), nacolomab (tafenatox), naptumomab (estafenatox), narnatumab, necitumumab, nimotuzumab, nivolumab, Neuradiab® (with or without radioactive iodine), NR-LU-10, ofatumumab, olaratumab, onartuzumab, oportuzumab (monatox), oregovomab, panitumumab, patritumab, pemtumomab, pertuzumab, pritumumab, racotumomab, radretumab, ramucirumab, rilotumumab, rituximab, robatumumab, samalizumab, sibrotuzumab, siltuximab, tabalumab, taplitumomab (paptox), tenatumomab, teprotumumab, TGN1412, ticilimumab, tremelimumab, tigatuzumab, TNX-650, tositumomab, TRBS07, trastuzumab, tucotuzumab (celmoleukin), ublituximab, urelumab, veltuzumab, volociximab, votumumab, and zalutumumab. Also included are fragments, variants, and derivatives of these antibodies.

The methods described herein can be used in the treatment and/or diagnosis of any variety of cancers or tumors. In some embodiments, the cancer is a primary cancer, i.e., a cancer growing at the anatomical site where tumor progression began and yielded a cancerous mass. In some embodiments, the cancer is a secondary or metastatic cancer, i.e., a cancer which has spread from the primary site or tissue of origin into one or more different sites or tissues. In some instances, the cancer is selected from one or more of carcinoma, sarcoma such as rhabdomyosarcoma, for example, alveolar rhabdomyosarcoma (including sarcoma originating in the bones, tendons, cartilage, muscle, fat, fibrous, blood vessels, adipose, and/or connective tissue), neuroblastoma, medulloblastoma, astrocytoma, glioblastoma multiforme, retinoblastoma, myeloma, leukemia, lymphoma (including Hodgkin's lymphoma and Non-Hodgkin's lymphoma), adenosquamous carcinoma, carcinosarcoma, mixed mesodermal tumor, teratocarcinoma, lung cancer (including non-small cell lung cancer, small cell lung cancer, adenocarcinoma, and squamous carcinoma of the lung), breast cancer (including metastatic breast cancer), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

In specific embodiments, the MYC gene is MYCC and the cancer is selected from lung cancers and blood cancers, optionally leukemias and lymphomas. In specific embodiments, the MYC gene is MYCN and the cancer is selected from neuroblastoma, small cell lung cancer, prostate cancer, alveolar rhabdomyosarcoma, medulloblastoma, glioblastoma multiforme, retinoblastoma, and breast cancer.

In some embodiments, as noted above, the cancer or tumor is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

In certain embodiments, the methods described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods described are sufficient to result in stable disease. In certain embodiments, the methods described herein are sufficient to result in clinically relevant reduction in symptoms of a particular disease indication known to the skilled clinician.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, for instance, for the treatment of human disease or testing, the agents described herein are generally incorporated into one or more therapeutic or pharmaceutical compositions prior to administration.

To prepare a therapeutic or pharmaceutical composition, an effective or desired amount of one or more agents is typically mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Certain embodiments include the use of a diagnostic kit for determining or predicting a therapeutic response (or responsiveness) to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] therapy in a subject with cancer, comprising means for measuring MYC expression level, MYC gene copy number, or MYC gene chromosomal location site, in a sample of tissue from the subject, including cancer tissue and non-cancerous tissue. Also included are patient care kits, comprising: (a) means for measuring MYC expression level, MYC gene copy number, or MYC gene chromosomal location site, in a sample of tissue from a subject, including cancer tissue and non-cancerous tissue; (b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide]; and (c) a second anti-cancer agent, wherein cancer cell-killing activity of the second anti-cancer agent occurs predominantly in the M phase and/or G1 phase of the cell cycle, as described herein.

In some embodiments, the means for measuring MYC expression level or MYC gene copy number comprise reagents for performing a diagnostic assay selected from one or more of Western blot, in situ hybridization (ISH), fluorescence in situ hybridization (FISH), enzyme-linked immunosorbent assay (ELISA), array comparative genome hybridization (aCGH), single nucleotide polymorphism (SNP) array, copy number variation (CNV) sequencing, and multiplex ligation-dependent probe amplification (MLPA) on a human MYC gene. In some embodiments, the means for measuring MYC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYC gene.

Certain diagnostic or patient care kits include a MYC expression level or MYC gene copy number reference value obtained from a database, or determined from a non-cancerous tissue from a control. Some diagnostic or patient care kits include a MYC expression level or MYC gene chromosomal location site reference obtained from a database, or determined from a non-cancerous tissue from a control. The kits can also include written instructions, for example, on how to determine MYC expression level, MYC gene copy number, and/or a MYC gene chromosomal location site in a sample of cancer tissue from a subject, and/or from a non-cancerous control.

In some embodiments, a diagnostic or patient care kit contains separate containers, dividers, or compartments for the composition(s) and informational material(s). For example, the composition(s) or reagents can be contained in a bottle, vial, or syringe, and the informational material(s) can be contained in association with the container. In some embodiments, the separate elements of the kit are contained within a single, undivided container. For example, the composition(s) or reagents are contained in a bottle, vial or syringe that has attached thereto the informational material in the form of a label. In some embodiments, the kit includes a plurality (e.g., a pack) of individual containers, each containing one or more compositions, reagents, and/or unit dosage forms of YM155 monobromide. For example, the kit includes a plurality of syringes, ampules, foil packets, or blister packs, each containing a reagent or a single unit dose of YM155 monobromide. The containers of the kits can be air tight, waterproof (e.g., impermeable to changes in moisture or evaporation), and/or light-tight.

The patient care kit optionally includes a device suitable for administration of the agent(s), e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

In certain aspects, the diagnostic or therapeutic response tests or methods described herein are performed at a diagnostic laboratory, and the results are then provided to the subject, or to a physician or other healthcare provider that plays a role in the subject's healthcare and cancer treatment. Particular embodiments thus include methods for providing the results of the responsiveness test to the subject in need thereof, or to the physician or other healthcare provider. These results or data can be in the form of a hard-copy or paper-copy, or an electronic form, such as a computer-readable medium.

Also included are methods of screening an anti-cancer agent for use in combination with YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] for treating a MYC-associated cancer in a subject, comprising

(a) contacting a population of cancer cells with the second anti-cancer agent, wherein MYC expression level or MYC gene copy number in the cancer cells is increased relative to that of a MYC gene copy number reference, or wherein a MYC gene chromosomal location site in the cancer cells is translocated relative to that of a MYC gene chromosomal location site reference;

(b) measuring the amount of live:dead cancer cells in the M phase, G1 phase, and/or S/G2 phase of the cell cycle;

(c) characterizing or identifying the anti-cancer agent as effective for use in combination with YM155 monobromide if the cancer cell-killing activity of the anti-cancer agent occurs predominantly in the M phase or G1 phase of the cell cycle, and characterizing anti-cancer agent as not effective for use in combination with YM155 monobromide if the cancer cell-killing activity of the anti-cancer agent occurs predominantly in the S/G2 phase of the cell cycle.

In particular embodiments, the methods are performed in vitro, for example, in tissue culture with cancer cell lines and/or primary cancer cells. Cell death and cell cycle parameters can be characterized according to routine techniques in the art, for example, flow cytometry methods which provide the relative cell cycle distribution of a population of cells, and which can also identify cells undergoing apoptosis or necrosis (see, for example, Darzynkiewicz et al., 38(2):179-93, 2001), and the Examples section.

Examples of anti-cancer agents for use in methods of screening include, without limitation, chemotherapeutic agents, cancer immunotherapy agents, hormonal therapeutic agents, and kinase inhibitors, including combinations of the foregoing.

In some embodiments, the at least one chemotherapeutic agent is selected from one or more of an alkylating agent, an anti-metabolite, a cytotoxic antibiotic, a topoisomerase inhibitor (type 1 or type II), and an anti-microtubule agent.

In some embodiments, the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);

the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);

the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;

the topoisomerase inhibitor is selected from one or more of camptothecin, irinotecan, topotecan, etoposide, doxorubicin, mitoxantrone, teniposide, novobiocin, merbarone, and aclarubicin; and/or

the anti-microtubule agent is selected from one or more of taxanes (optionally paclitaxel and docetaxel) and vinca alkaloids (optionally vinblastine, vincristine, vindesine, vinorelbine).

In some embodiments, the cancer immunotherapy agent is selected from one or more of an immune checkpoint modulatory agent and a cytokine. In some embodiments, the immune checkpoint modulatory agent is a polypeptide, optionally an antibody or antigen-binding fragment thereof or a ligand, or a small molecule. In some embodiments, the immune checkpoint modulatory agent comprises

(a) an antagonist of a inhibitory immune checkpoint molecule; or

(b) an agonist of a stimulatory immune checkpoint molecule, for example, wherein the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736);

the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab;

the antagonist is a CTLA-4 antagonist optionally selected from one or more of an antibody or antigen-binding fragment or small molecule that specifically binds thereto, ipilimumab, tremelimumab;

the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat;

the antagonist is a TDO antagonist optionally selected from one or more of an antibody or antigen-binding fragment or small molecule that specifically binds thereto, 680C91, and LM10;

the antagonist is a TIM-3 antagonist optionally selected from one or more of an antibody or antigen-binding fragment or small molecule that specifically binds thereto;

the antagonist is a LAG-3 antagonist optionally selected from one or more of an antibody or antigen-binding fragment or small molecule that specifically binds thereto, and BMS-986016;

the antagonist is a VISTA antagonist optionally selected from one or more of an antibody or antigen-binding fragment or small molecule that specifically binds thereto;

the antagonist is a BTLA, CD160, and/or HVEM antagonist optionally selected from one or more of an antibody or antigen-binding fragment or small molecule that specifically binds thereto;

the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agonist is an OX40 agonist optionally selected from one or more of an antibody or antigen-binding fragment or small molecule or ligand that specifically binds thereto, OX86, Fc-OX40L, and GSK3174998;

the agonist is a CD40 agonist optionally selected from one or more of an antibody or antigen-binding fragment or small molecule or ligand that specifically binds thereto, CP-870,893, dacetuzumab, Chi Lob 7/4, ADC-1013, and rhCD40L;

the agonist is a GITR agonist optionally selected from one or more of an antibody or antigen-binding fragment or small molecule or ligand that specifically binds thereto, INCAGN01876, DTA-1, and MEDI1873;

the agonist is a CD137 agonist optionally selected from one or more of an antibody or antigen-binding fragment or small molecule or ligand that specifically binds thereto, utomilumab, and 4-1BB ligand;

the agonist is a CD27 agonist optionally selected from one or more of an antibody or antigen-binding fragment or small molecule or ligand that specifically binds thereto, varlilumab, and CDX-1127 (1F5);

the agonist is a CD28 agonist optionally selected from one or more of an antibody or antigen-binding fragment or small molecule or ligand that specifically binds thereto, and TAB08; and/or

the agonist is an HVEM agonist optionally selected from one or more of an antibody or antigen-binding fragment or small molecule or ligand that specifically binds thereto.

In some embodiments, the cytokine selected from one or more of interferon (IFN)-α, IL-2, IL-12, IL-7, IL-21, and Granulocyte-macrophage colony-stimulating factor (GM-CSF).

In some embodiments, the at least one hormonal therapeutic agent is a hormonal agonist or a hormonal antagonist. In some embodiments, the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog. In some embodiments, the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib. In some embodiments, the kinase inhibitor is a PI3 kinase inhibitor selected from one or more of alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147.

Certain embodiments comprise characterizing, identifying, and/or selecting the anti-cancer agent as effective for use in combination with YM155 monobromide if at least 60, 65, 70, 75, 80, 85, 90, or 95% of the cancer cell-killing activity of the anti-cancer agent occurs in the M phase of the cell cycle. Some embodiments comprise characterizing, identifying, and/or selecting the anti-cancer agent as effective for use in combination with YM155 monobromide if at least 60, 65, 70, 75, 80, 85, 90, or 95% of the cancer cell-killing activity of the anti-cancer agent occurs in the G1 phase of the cell cycle.

Certain embodiments further comprise (d) contacting a population of cancer cells (optionally in vitro) with YM155 in combination with the identified or selected anti-cancer agent from (c), wherein MYC gene copy number in the cancer cells is increased relative to that of a MYC gene copy number reference, or wherein a MYC gene chromosomal location site in the cancer cells is translocated relative to that of a MYC gene chromosomal location site reference; (e) measuring tumor cell proliferation and/or tumor cell apoptosis in the population of cancer cells, optinally in combination with cell cycle analysis; and (f) characterizing or identifying or selecting the anti-cancer agent as optimal for use in combination with YM155 monobromide if the combined cancer cell-killing activity of the anti-cancer agent and YM155 is significantly (optionally synergistically) increased relative to that of YM155 and the anti-cancer agent alone. The term “synergistically” refers to more than an additive effect.

The cancer cell-killing activity can be evaluated or measured by any variety of techniques, including by the effects on cancer cell proliferation and/or cancer cell apoptosis. Methods of measuring tumor cell proliferation and/or tumor cell apoptosis are known in the art. For example, certain methods of measuring tumor cell proliferation include measuring one or more cellular proliferation markers. Exemplary cellular proliferation markers include ³H-thymidine, bromodeoxyuridine (BrdU), 5-ethynyl-2′-deoxyuridine (Edu), Ki-67, and proliferating cell nuclear antigen (PCNA). Thus, in certain embodiments, the step of measuring tumor cell proliferation comprises measuring a cellular proliferation marker, which is optionally selected from one or more of ³H-thymidine, BrdU, Edu, Ki-67, and PCNA, including combinations thereof.

Likewise, any variety of methods known in the art can be used to measure tumor cell apoptosis. “Apoptosis” refers generally to a process of programmed cell death that occurs in multicellular organisms, including biochemical events that lead to characteristic cell changes (e.g., morphology) and cell death. Exemplary changes include blebbing, cell shrinkage, nuclear fragmentation, chromatin condensation, chromosomal DNA fragmentation, and global mRNA decay. Certain methods of measuring tumor cell apoptosis include measuring a cellular apoptosis marker. Exemplary cellular apoptosis markers include fluorochrome-labeled inhibitors of Caspases (FLICA), caspase activation, poly ADP ribose polymerase (PARP) cleavage, DRAQ5, DRAQ7, and a terminal deoxynucleotidyl transferase (TdT) dUTP nick-end labeling (TUNEL) assay. Thus, in certain embodiments, the step of measuring tumor cell apoptosis comprises measuring a cellular apoptosis marker, which is optionally selected from one or more of FLICA, PARP, DRAQ5, DRAQ7, and a TUNEL assay, including combinations thereof.

In some embodiments, the population of cancer cells is selected from one or more of neuroblastoma, carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), radiation-induced angiosarcoma, medulloblastoma, astrocytoma, glioblastoma multiforme, retinoblastoma, myeloma, leukemia, lymphoma (including Hodgkin's lymphoma and Non-Hodgkin's lymphoma such as diffuse large B-cell lymphomas), adenosquamous carcinoma, carcinosarcoma, mixed mesodermal tumor, teratocarcinoma), lung cancer (including non-small cell lung cancer, small cell lung cancer, adenocarcinoma, and squamous carcinoma of the lung), breast cancer (including metastatic breast cancer), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer. In some embodiments, the population of cancer cells is homogenous, for example, as a cancer cell line. In some embodiments, the population of cancer cells are primary cancer cells.

In some embodiments, the MYC gene is selected from MYCN and MYCC. In particular embodiments, the MYC gene is MYCN and the population of cancer cells is selected from neuroblastoma, small cell lung cancer, prostate cancer, alveolar rhabdomyosarcoma, medulloblastoma, glioblastoma multiforme, retinoblastoma, and breast cancer cells, including cell lines and primary cells of the foregoing. In some embodiments, the MYC gene is MYCC and the population of cancer cells is selected from lung cancers, optionally non-small lung cell cancer, blood cancers, optionally leukemias and lymphomas such as diffuse large B-cell lymphomas, and sarcomas, for example, radiation-induced angiosarcoma cells, including cell lines and primary cells of the foregoing.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

### Cell Cycle-Related Cell-Killing Activity of YM155

Studies were performed to evaluate the efficacy of YM155 monobromide in various cancer cell lines, and also to correlate the its cancer cell-killing activity to the cell cycle, relative to that of other anti-cancer agents such as CHOP (cyclophosphamide, doxorubicin, vincristine, and prednisone).

Materials & Methods

Cell Culture Human neuroblastoma cell line SHEP-MYCN-ER cells were cultured in RPMI 1640 (Hyclone™, SH30809.01B) supplemented with 10% fetal bovine serum (GEMINI, 900-108) with 200 nM 4-OHT (4-hydroxytamoxifen, Sigma-Aldrich). 4-OHT treatment induced high MYCN expression. Cultures were incubated at 37° C. in 5% CO₂.

Cell transfection. Plate SHEP-MYCN-ER cells in 96-well plates (Corning-Costar, 3599) at 2000 cells/well and allow 4 hours for cells to adhere. Mix each FUCCI Premo™ reagent (Thermo fisher, P36237) by inversion to ensure a homogenous solution. Add the mixed reagent to the SHEP-MYCN-ER cells in complete cell medium and mix gently. Return the cells to the culture incubator for over 16 hour.

High-content live-cell imaging analysis. After transfection, SHEP-MYCN-ER cells were incubated in complete cell medium with concentration indicated of different drugs (PC-002: 10 nM YM155, CHOP: 2 μM cyclophosphamide; 0.2 μM Doxorubicin; 8 nM vincristine). Cells were grown in Array Scan™ Live Cell Module and imaged every 20 min for 72 h under controlled growth conditions (CellInsight™ CX5 High-Content Screening (HSC) Platform, Thermo Fisher; Array Scan™ Live Cell Module, Thermo Fisher).

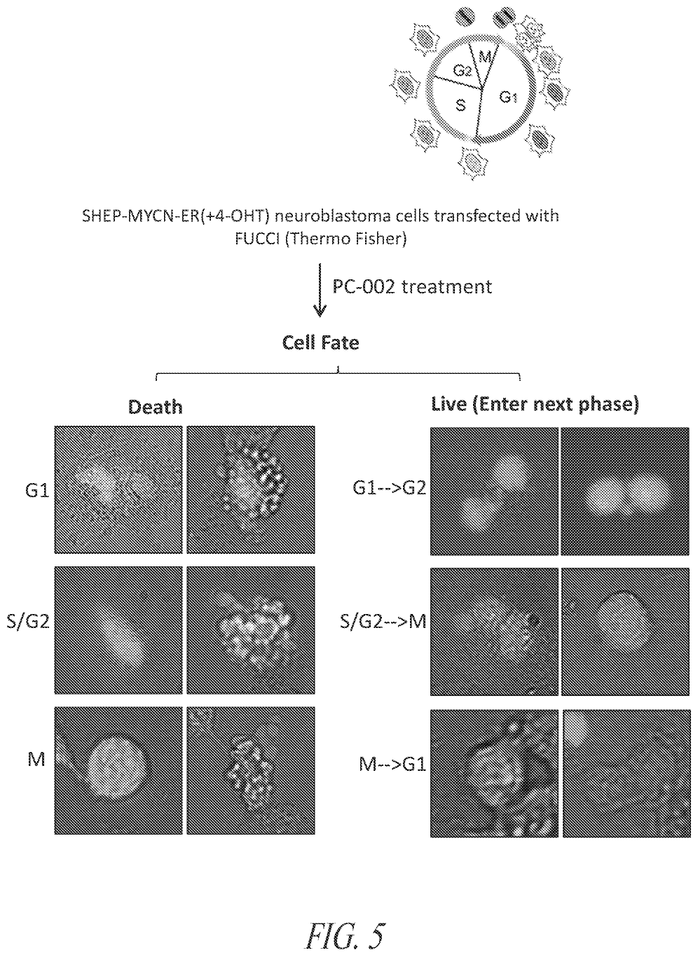

The fluorescence ubiquitination cell cycle indicator (FUCCI), a fluorescent protein (FP)-based sensor that employs a red (RFP) and a green (GFP) fluorescent protein fused to different regulators of the cell cycle: Cdtl and geminin Both constructs are ubiquitinated by specific ubiquitin E3 ligases targeting them to the proteasome for degradation. In the G1 phase of the cell cycle, geminin is broken down and only Cdtl tagged with RFP may be visualized, thus identifying cells in the G1 phase with red fluorescent nuclei. In the S, G2, and M phases, however, Cdt1 is degraded and only geminin tagged with GFP remains, thus identifying cells in these phases with green fluorescent nuclei. During the G1/S transition, both proteins are present in the cells, allowing GFP and RFP fluorescence to be observed—when green and red images are overlaid, the cells appear with yellow (orange) fluorescent nuclei. The dynamic color change from red-to-yellow, yellow-to-green or green-to-red represents the progression through cell cycle (live). The cell dead without color change represents the sensitivity to the drug (death). FIG. 5 illustrates the cell cycle-related fate of neuroblastoma cells following YM155 (PC-002) treatment.

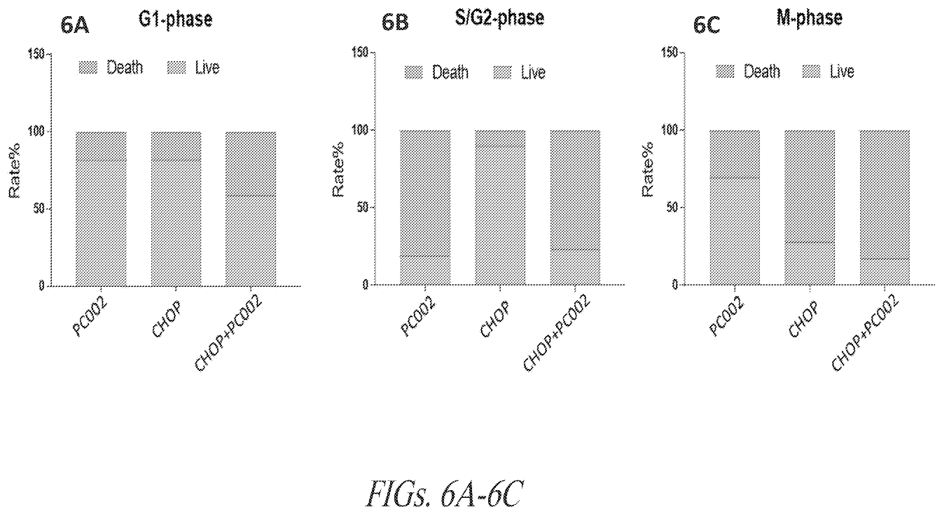

The results are shown in FIGS. 6A-6C. As shown in FIG. 6B, the cancer cell-killing activity of YM155 occurs predominantly in the S/G2 phase of the cell cycle, and not significantly in the G1 phase or M phase. In contrast, as shown in FIG. 6C, the cancer cell-killing activity of CHOP occurs predominantly in M-phase, and not significantly in the S/G2 phase or G1 phase.

FIGS. 6A-6B further illustrate that the cancer cell-killing activity of YM155 in combination with CHOP occurs mostly in both the S/G2 phase and M phase, resulting in a significant, overall increase in cancer cell death. This latter observation evidences that effective combination therapies for use with YM155 can be identified by the cell cycle-related activity of a given anti-cancer agent, particularly if cancer cell-killing activity of the anti-cancer agent occurs predominantly in the M phase or G1 phase of the cell cycle (i.e., not substantially in the S/G2 phase of the cell cycle).

1. A method for treating a MYC-associated cancer in a subject in need thereof, comprising
administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject in combination with a second anti-cancer agent, wherein cancer cell-killing activity of the second anti-cancer agent occurs predominantly in the M phase or G1 phase of the cell cycle,
thereby treating the MYC-associated cancer in the subject in need thereof.

2. The method of claim 1, comprising,
(a) determining MYC expression level, MYC gene copy number, or MYC gene chromosomal location site, in a sample of cancer tissue from the subject; and
(b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject in combination with the second anti-cancer agent if MYC expression level or MYC gene copy number in the cancer tissue is increased relative to that of a MYC expression level reference or MYC gene copy number reference, or if MYC gene chromosomal location site in the cancer tissue is translocated relative to that of a MYC gene chromosomal location site reference.

3. The method of claim 1 or 2, comprising administering to the subject a chemotherapeutic agent excluding (or other than) YM155 monobromide if MYC expression level or MYC gene copy number in the cancer tissue is not substantially increased relative to that of the MYC expression level reference or MYC gene copy number reference, or if MYC gene chromosomal location site in the cancer tissue is not translocated relative to that of the MYC gene chromosomal location site reference.

4. The method of claim 1 or 2, wherein at least 60, 65, 70, 75, 80, 85, 90, or 95% of the cancer cell-killing activity of the second anti-cancer agent occurs in the M phase of the cell cycle, optionally wherein the second anti-cancer agent is selected from one or more of vinca alkaloids and taxanes, or optionally selected from one or more of CHOP (cyclophosphamide, doxorubicin, vincristine, and prednisone), vinblastine, vincristine, vinorelbine, cabazitaxel, docetaxel, paclitaxel, eribulin, estramustine, and ixabepilone.

5. The method of claim 1 or 2, wherein at least 60, 65, 70, 75, 80, 85, 90, or 95% of the cancer cell-killing activity of the second anti-cancer agent occurs in the G1 phase of the cell cycle, optionally wherein the second anti-cancer agent is selected from one or more of mitomycin, asparaginase, and pegaspargase.

6. The method of any one of claims 1-5, wherein the MYC expression level or MYC gene copy number in the cancer tissue is increased by about or at least about 1.5, 2, 3, 4, 5, 6, 7, 8, 9, or 10-fold relative to that of the MYC expression level reference or MYC gene copy number reference.

7. The method of any one of claims 1-6, comprising determining MYC expression level or MYC gene copy number in the cancer tissue by Western blot, in situ hybridization (ISH), fluorescence in situ hybridization (FISH), enzyme-linked immunosorbent assay (ELISA), array comparative genome hybridization (aCGH), single nucleotide polymorphism (SNP) array, copy number variation (CNV) sequencing, or multiplex ligation-dependent probe amplification (MLPA).

8. The method of any one of claims 1-7, comprising determining MYC gene chromosomal location site in the cancer tissue by in situ hybridization (ISH), fluorescence in situ hybridization (FISH), next generation sequencing (NGS), or comparative genome hybridization (CGH).

9. The method of any one of claims 1-8, comprising obtaining the MYC expression level or MYC gene copy number reference from a database, or determining the MYC expression level or MYC gene copy number reference from a non-cancerous tissue from a control, optionally by Western blot, ISH, FISH, ELISA, aCGH, SNP array, CNV sequence, or MLPA.

10. The method of any one of claims 1-9, comprising obtaining the MYC gene chromosomal location site reference from a database, or determining the MYC gene chromosomal location site reference from a non-cancerous tissue from a control, optionally by ISH, FISH, NGS, or CGH.

11. The method of any one of claims 1-10, comprising obtaining the sample of cancer tissue from the subject.

12. The method of any one of claims 1-11, wherein the sample of cancer tissue is a surgical sample, a biopsy sample, a pleural effusion sample, or an ascetic fluid sample obtained from the subject, optionally selected from one or more of lung, blood, breast, gastrointestinal (stomach, colon, rectal), ovarian, pancreatic, liver, bladder, cervical, neuronal, uterine, salivary gland, kidney, prostate, thyroid, or muscle tissue.

13. The method of any one of claims 1-12, wherein the subject is a human subject.

14. The method of any one of claims 1-13, wherein the cancer is selected from one or more of neuroblastoma, carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), radiation-induced angiosarcoma, medulloblastoma, astrocytoma, glioblastoma multiforme, retinoblastoma, myeloma, leukemia, lymphoma (including Hodgkin's lymphoma and Non-Hodgkin's lymphoma such as diffuse large B-cell lymphomas), adenosquamous carcinoma, carcinosarcoma, mixed mesodermal tumor, teratocarcinoma), lung cancer (including non-small cell lung cancer, small cell lung cancer, adenocarcinoma, and squamous carcinoma of the lung), breast cancer (including metastatic breast cancer), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

15. The method of any one of claims 1-14, wherein the MYC gene is selected from MYCN and MYCC.

16. The method of claim 15, wherein the MYC gene is MYCN and the cancer is selected from neuroblastoma, small cell lung cancer, prostate cancer, alveolar rhabdomyosarcoma, medulloblastoma, glioblastoma multiforme, retinoblastoma, and breast cancer.

17. The method of claim 15, wherein the MYC gene is MYCC and the cancer is selected from lung cancers, optionally non-small lung cell cancer, blood cancers, optionally leukemias and lymphomas such as diffuse large B-cell lymphomas, and sarcomas, optionally radiation-induced angiosarcomas.

18. The method of any one of claim 1-2 or 4-17, wherein YM155 and the second anti-cancer agent are administered separately or sequentially.

19. The method of any one of claim 1-2 or 4-17, wherein YM155 and the second anti-cancer agent are administered together at the same time.

20. A method of screening an anti-cancer agent for use in combination with YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] for treating a MYC-associated cancer in a subject, comprising
(a) contacting a population of cancer cells (optionally in vitro) with the anti-cancer agent, wherein MYC expression level or MYC gene copy number in the cancer cells is increased relative to that of a MYC expression level reference or a MYC gene copy number reference, or wherein a MYC gene chromosomal location site in the cancer cells is translocated relative to that of a MYC gene chromosomal location site reference;
(b) measuring the amount of live:dead cancer cells in the M phase, G1 phase, and/or S/G2 phase of the cell cycle;
(c) characterizing or identifying or selecting the anti-cancer agent as effective for use in combination with YM155 monobromide if the cancer cell-killing activity of the anti-cancer agent occurs predominantly in the M phase or G1 phase of the cell cycle, and characterizing anti-cancer agent as not effective for use in combination with YM155 monobromide if the cancer cell-killing activity of the anti-cancer agent occurs predominantly in the S/G2 phase of the cell cycle.

21. The method of claim 20, wherein the second anti-cancer agent is selected from one or more of chemotherapeutic agents, cancer immunotherapy agents, hormonal therapeutic agents, and kinase inhibitors, including combinations of the foregoing.

22. The method of claim 21, wherein the chemotherapeutic agent is selected from one or more of an alkylating agent, an anti-metabolite, a cytotoxic antibiotic, a topoisomerase inhibitor (type 1 or type II), and an anti-microtubule agent.

23. The method of claim 22, wherein the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);
the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);
the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;
the topoisomerase inhibitor is selected from one or more of camptothecin, irinotecan, topotecan, etoposide, doxorubicin, mitoxantrone, teniposide, novobiocin, merbarone, and aclarubicin; and/or
the anti-microtubule agent is selected from one or more of taxanes (optionally paclitaxel and docetaxel) and vinca alkaloids (optionally vinblastine, vincristine, vindesine, vinorelbine).

24. The method of claim 21, wherein the cancer immunotherapy agent is an antagonist of an inhibitory immune checkpoint molecule selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT); optionally wherein the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736); optionally wherein the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab; optionally wherein the antagonist is a CTLA-4 antagonist optionally selected from one or more of an antibody or antigen-binding fragment or small molecule that specifically binds thereto, ipilimumab, and tremelimumab.

25. The method of claim 21, wherein the cancer immunotherapy agent is an agonist of a stimulatory immune checkpoint molecule selected from one or more of OX40, CD40, Glucocorticoid-Induced TNFR Family Related Gene (GITR), CD137 (4-1BB), CD27, CD28, CD226, and Herpes Virus Entry Mediator (HVEM).

26. The method of claim 21, wherein the cancer immunotherapy agent is a cytokine selected from one or more of interferon (IFN)-α, IL-2, IL-12, IL-7, IL-21, and Granulocyte-macrophage colony-stimulating factor (GM-CSF).

27. The method of claim 21, wherein the hormonal therapeutic agent is a hormonal agonist or a hormonal antagonist, optionally wherein the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog, optionally wherein the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

28. The method of claim 21, wherein the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib, optionally wherein the kinase inhibitor is a PI3 kinase inhibitor selected from one or more of alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147.

29. The method of any one of claims 20-28, comprising characterizing or identifying or selecting the anti-cancer agent as effective for use in combination with YM155 monobromide if at least 60, 65, 70, 75, 80, 85, 90, or 95% of the cancer cell-killing activity of the anti-cancer agent occurs in the M phase of the cell cycle.

30. The method of any one of claims 20-29, comprising characterizing or identifying or selecting the anti-cancer agent as effective for use in combination with YM155 monobromide if at least 60, 65, 70, 75, 80, 85, 90, or 95% of the cancer cell-killing activity of the anti-cancer agent occurs in the G1 phase of the cell cycle.

31. The method of any one of claims 20-30, further comprising (d) contacting a population of cancer cells (optionally in vitro) with YM155 in combination with the identified or selected anti-cancer agent from (c), wherein MYC expression level or MYC gene copy number in the cancer cells is increased relative to that of a MYC expression level reference or MYC gene copy number reference, or wherein a MYC gene chromosomal location site in the cancer cells is translocated relative to that of a MYC gene chromosomal location site reference;
(e) measuring tumor cell proliferation and/or tumor cell apoptosis in the population of cancer cells; and
(f) characterizing or identifying or selecting the anti-cancer agent as optimal for use in combination with YM155 monobromide if the combined cancer cell-killing activity of the anti-cancer agent and YM155 is significantly (optionally synergistically) increased relative to that of YM155 and the anti-cancer agent alone.

32. The method of any one of claims 20-31, wherein the population of cancer cells is selected from one or more of neuroblastoma, carcinoma, sarcoma such as rhabdomyosarcoma for example, alveolar rhabdomyosarcoma, (including sarcoma originating in the bones, tendons, cartilage, muscle, fat, fibrous, blood vessels, adipose, and/or connective tissue), radiation-induced angiosarcoma, medulloblastoma, astrocytoma, glioblastoma multiforme, retinoblastoma, myeloma, leukemia, lymphoma (including Hodgkin's lymphoma and Non-Hodgkin's lymphoma such as diffuse large B-cell lymphomas), adenosquamous carcinoma, carcinosarcoma, mixed mesodermal tumor, teratocarcinoma), lung cancer (including non-small cell lung cancer, small cell lung cancer, adenocarcinoma, and squamous carcinoma of the lung), breast cancer (including metastatic breast cancer), gastrointestinal cancer, stomach cancer, colorectal cancer, colon cancer, rectal cancer, ovarian cancer, pancreatic cancer, liver cancer, bladder cancer, cervical cancer, glioblastoma, uterine carcinoma, salivary gland carcinoma, kidney or renal cancer (e.g., Wilm's tumor), prostate cancer, thyroid cancer, and head and neck cancer.

33. The method of any one of claims 20-32, wherein the MYC gene is selected from MYCN and MYCC.

34. The method of claim 33, wherein the MYC gene is MYCN and the population of cancer cells is selected from neuroblastoma, small cell lung cancer, prostate cancer, alveolar rhabdomyosarcoma, medulloblastoma, glioblastoma multiforme, retinoblastoma, and breast cancer.

35. The method of claim 33, wherein the MYC gene is MYCC and the population of cancer cells is selected from lung cancers, optionally non-small lung cell cancer, blood cancers, optionally leukemias and lymphomas such as diffuse large B-cell lymphomas, and sarcomas, optionally radiation-induced angiosarcomas.

36. A patient care kit, comprising:
(a) means for measuring MYC expression level, MYC gene copy number, or MYC gene chromosomal location site, in a sample of tissue from a subject, including cancer tissue and non-cancerous tissue;
(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof; and
(c) a second anti-cancer agent, wherein cancer cell-killing activity of the second anti-cancer agent occurs predominantly in the M phase and/or G1 phase of the cell cycle.

37. The patient care kit of claim 36, wherein the means for measuring MYC expression level or MYC gene copy number comprise reagents for performing a diagnostic assay selected from one or more of Western blot, in situ hybridization (ISH), fluorescence in situ hybridization (FISH), enzyme linked immunosorbent assay (ELISA), array comparative genome hybridization (aCGH), single nucleotide polymorphism (SNP) array, copy number variation (CNV) sequencing, and multiplex ligation-dependent probe amplification (MLPA) on a human MYC gene.

38. The patient care kit of claim 36 or 37, wherein the means for measuring MYC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYC gene.

39. The patient care kit of any one of claims 36-38, comprising a MYC expression level reference or a MYC gene copy number reference value obtained from a database, or determined from a non-cancerous tissue from a control.

40. The patient care kit of any one of claims 36-39, comprising a MYC gene chromosomal location site reference obtained from a database, or determined from a non-cancerous tissue from a control.

41. The patient care kit of any one of claims 36-40, wherein the MYC gene is selected from MYCC and MYCN.

42. The patient care kit of any one of claims 36-41, wherein at least 60, 65, 70, 75, 80, 85, 90, or 95% of the cancer cell-killing activity of the second anti-cancer agent occurs in the M phase of the cell cycle, optionally wherein the second anti-cancer agent is selected from one or more of vinca alkaloids and taxanes, or optionally selected from one or more of CHOP (cyclophosphamide, doxorubicin, vincristine, and prednisone), vinblastine, vincristine, vinorelbine, cabazitaxel, docetaxel, paclitaxel, eribulin, estramustine, and ixabepilone.

43. The patient care kit of any one of claims 36-42, wherein at least 60, 65, 70, 75, 80, 85, 90, or 95% of the cancer cell-killing activity of the second anti-cancer agent occurs in the G1 phase of the cell cycle, optionally wherein the second anti-cancer agent is selected from one or more of mitomycin, asparaginase, and pegaspargase.
